(19) United States
(12) Patent Application Publication
(10) Pub. No.: US 2021/0330835 A1
(43) Pub. Date: Oct. 28, 2021

(54) ULTRAVIOLET RADIATION DISINFECTION SYSTEM AND METHOD

(71) Applicant: Barry Hunt, Cambridge (CA)

(72) Inventor: Barry Hunt, Cambridge (CA)

(21) Appl. No.: 17/240,353

(22) Filed: Apr. 26, 2021

Related U.S. Application Data
(60) Provisional application No. 63/014,988, filed on Apr. 24, 2020.

(51) Int. Cl. A61L 2/10; A61L 2/24

(52) U.S. Cl. CPC A61L 2/10; A61L 2/24; A61L 2202/26; A61L 2202/11; A61L 2202/122

(57) ABSTRACT

A disinfection system for disinfecting an object using ultraviolet radiation, including a conveyor assembly operable to carry the object along a path, the path defining a conveying plane; a support frame supporting the conveyor assembly, the support frame including a housing covering at least a portion of the path; an ultraviolet radiation system mounted to the support frame and operable to irradiate the object with ultraviolet radiation from at least two independent positions as the object is carried along the path within the housing, the at least two independent positions including a first position adjacent a first side of the conveying plane and a second position adjacent a second side of the conveying plane across the conveying plane from the first side.

### FIELD

The disclosure relates to decontamination, particularly to an apparatus and method for disinfection using ultraviolet radiation.

### BACKGROUND

U.S. Pat. No. 8,624,203 to Tullo et al. (“Tullo”) purports to disclose a sterilizing system for sterilizing a continuous loop conveyor belt of a conveyor assembly is provided. Tullo purports to disclose a conveyor assembly that includes a drive operatively connected to the conveyor belt and operative to move the belt between upper and lower flight paths, the upper flight path including an exposed surface for receiving items, the sterilizing system including a housing that is configured to at least partially cover the lower flight path, an ultraviolet light source positioned in the interior of the housing, and the light source operative to emit ultraviolet light on the belt at the lower flight path to sanitize the belt.

U.S. Pat. No. 8,975,605 to Neister (“Neister”) purports to disclose an improved method, process and apparatus for disinfecting and sterilizing all types of surfaces and indoor air and room air contaminated with microorganisms. Neister purports to disclose an improved apparatus that consists of a multi-wavelength narrow spectral width UV source that is more effective than mercury based 254 nm germicidal lamps for destroying the DNA and outer shell or membrane of virus, bacteria, spores and cists.

### SUMMARY

In some aspects, there is provided a disinfection system for disinfecting an object using ultraviolet radiation, comprising a conveyor assembly operable to carry the object along a path, the path defining a conveying plane; a support frame supporting the conveyor assembly; an ultraviolet radiation system mounted to the support frame and operable to irradiate the object with ultraviolet radiation from at least two independent positions as the object is carried along the path, the at least two independent positions including a first position adjacent a first side of the conveying plane and a second position adjacent a second side of the conveying plane across the conveying plane from the first side.

In some examples, the support frame includes a housing covering at least a portion of the path, and the ultraviolet radiation system is operable to irradiate the object with ultraviolet radiation from the at least two independent positions as the object is carried along the path within the housing.

In some examples, the housing includes a tunnel enclosure through which the path extends.

In some examples, at least one inner surface of the housing is a reflective surface.

In some examples, the ultraviolet radiation system includes at least two independent radiation sources.

In some examples, each of the at least two independent radiation sources is a ultraviolet C lamp operable to generate ultraviolet C radiation.

In some examples, the conveyor assembly includes an endless loop conveyor forming a receiving surface to carry the object along the path.

In some examples, the disinfection system further comprises a control system operable to adjust at least one of a speed at which the conveyor assembly carries the object along the path and a distance between the ultraviolet radiation system and the object as the object is carried along the path,

In some examples, the control system is operable to receive an indication of an object type of the object and match at least one of the speed and the distance to the object type.

In some examples, the control system includes an imaging system operable to generate an image of the object, and the control system is operable to process the image to produce the indication of the object type.

In some examples, the object includes a removable compartment tray that may be removably positioned on the conveyor assembly to be carried along the path while holding at least one item in a compartment of the removeable compartment tray.

In some examples, the object includes a face mask, and the face mask is mounted to a removeable fixation tray, and the removable fixation tray is operable to carry the face mask in a fixed and opened configuration.

In some examples, the removable tray includes a plurality of fasteners to hold the face mask at a plurality of separate points on the face mask, the plurality of fasteners spaced from one another to hold the face mask in the opened configuration.

In some examples, the ultraviolet radiation system is operable to deliver at least 250 millijoules of ultraviolet C radiation per square centimeter to each of at least two opposite surfaces of the object.

In some examples, the conveyor assembly is operable to reposition the object at least once as the object travels along the path.

In some examples, the conveyor assembly includes at least one of a jostling system operable to jostle the object as the object travels along the path and a blocking system operable to block movement of the object along the path for a predetermined blocking time to reposition the object relative to the conveyor assembly.

In some examples, the object includes a face mask and the ultraviolet radiation system is operable to deliver ultraviolet radiation to at least two opposite surfaces of the face mask, the at least two opposite surfaces of the face mask including an exterior surface of the face mask and an interior surface of the face mask.

In some aspects, there is provided a disinfection method for disinfecting an object using ultraviolet radiation, comprising placing the object on a conveyor assembly; conveying the object along a path using the conveyor assembly, the path defining a conveying plane; and delivering ultraviolet C radiation to the object from at least two independent positions as the object is conveyed along the path, the at least two independent positions including a first position adjacent a first side of the conveying plane and a second position adjacent a second side of the conveying plane across the conveying plane from the first side.

In some examples, the disinfection method further comprises placing an indicator material adjacent the object prior to conveying the object along the path using the conveyor assembly; conveying the indicator material with the object along the path; and verifying, following delivering ultraviolet C radiation to the object, that the indicator material shows that a minimum quantity of ultraviolet C radiation was delivered.

In some examples, at least a portion of the path is covered by a housing, and the ultraviolet C radiation is delivered to the object from the at least two independent positions as the object is conveyed along the path within the housing.

### BRIEF DESCRIPTION OF THE DRAWINGS

The drawings included herewith are for illustrating various examples of systems, methods, and apparatus of the present specification. In the drawings:

FIG. 1 is a perspective view of a disinfection system;

FIG. 2 is a perspective view of a lamp of the disinfection system of FIG. 1;

FIG. 3 is a perspective view of a conveyor assembly of the disinfection system of FIG. 1;

FIG. 4 is a top view of a tray;

FIG. 5 is a side view of the tray of FIG. 4;

FIG. 6 is a side perspective view of the disinfection system of FIG. 1 with two of the trays of FIG. 4;

FIG. 7 is a flow chart of a method of disinfecting;

FIG. 8 is a side view of a bin;

FIG. 9 is a top view of the bin of FIG. 8;

FIG. 10 is a first perspective view of the bin of FIG. 8 adjacent the disinfection system of FIG. 1; and

FIG. 11 is a second perspective view of the bin of FIG. 8 adjacent the disinfection system of FIG. 1.

### DETAILED DESCRIPTION

Various apparatus or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

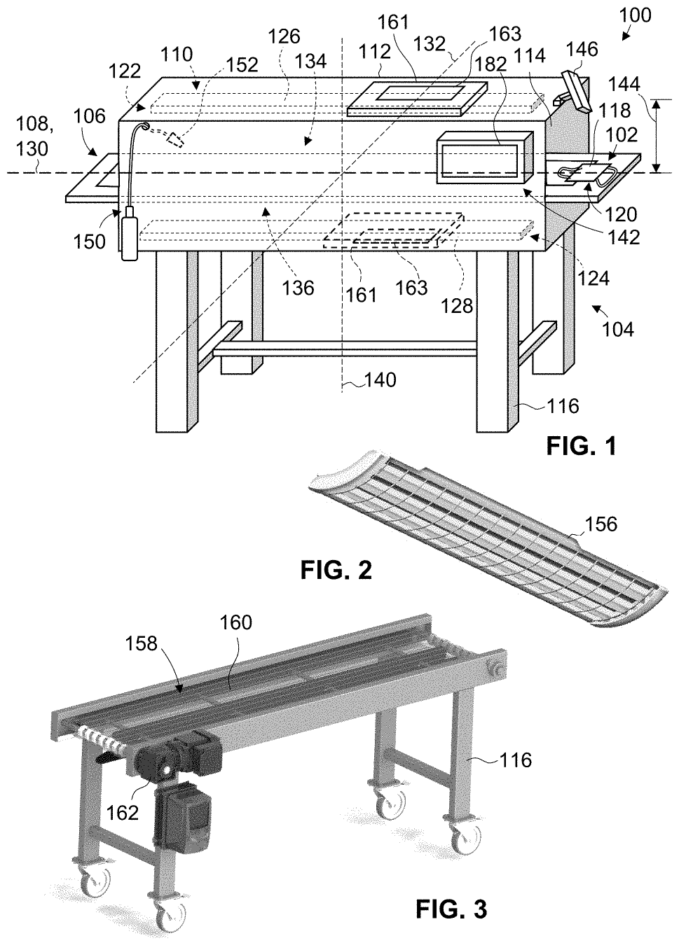

Referring to FIG. 1, illustrated is an example of a disinfection system 100 for disinfecting an object 102 using ultraviolet radiation. The object 102 may be, for example, contaminated with a pathogen such as a virus or a bacteria. The disinfection system 100 may deliver ultraviolet radiation, such as ultraviolet C radiation having a wavelength between 100 nanometers and 280 nanometers. The ultraviolet radiation may damage and/or kill the pathogen.

The disinfection system 100 includes a support frame 104. A conveyor assembly 106 is supported by to the support frame 104. The conveyor assembly 106 is operable to carry the object 102 along a path 108. An ultraviolet radiation system 110 is also mounted to the support frame.

In the example of FIG. 1, the support frame 104 includes a housing 112 covering at least a portion of the path 108. In the illustrated example, the housing 112 encloses the ultraviolet radiation system 110 and a portion of the conveyor assembly 106.

In some examples, the housing 112 shields adjacent individuals from ultraviolet light. In some examples, the housing 112 assists in directing ultraviolet radiation at the object. For example, an interior surface 114 of the enclosure housing 112 may be reflective, such as formed of a reflective aluminum, to direct ultraviolet radiation towards the object 102 as the object 102 is conveyed along the path 108.

In the illustrated example, the path 108 passes through the housing 112. The housing 112 forms a tunnel enclosure around a portion of the path 108.

In some examples, the enclosure housing 112 or a portion of the enclosure housing 112 is moveable relative to a base 116 of the support frame 104. For example, the enclosure housing 112 may move up and down relative to the base 116 to bring items mounted to the enclosure housing 112 farther from or closer to items mounted to the base 116.

The illustrated example ultraviolet radiation system 110 mounted to the support frame 104 is operable to irradiate at least two opposite surfaces of the object 102 with ultraviolet radiation as the object 102 is carried along the path 108 within the housing 112. In some examples, irradiating at least two opposite surfaces of the object 102 with ultraviolet radiation as the object 102 is carried along the path 108 assists in disinfecting the object 102 in one operation of the disinfection system 100.

In the illustrated example, the object 102 includes a first surface 118 and a second surface 120 opposite the first surface 118. In some examples, the ultraviolet radiation system 110 is operable to irradiate the first surface 118 and the second surface 120 with ultraviolet radiation as the object 102 is carried along the path 108.

Referring to FIGS. 1 to 6, the object 102 may be and/or include a face mask object, as illustrated. In some examples, the first surface 118 is an interior surface of the face mask object 102 and the second surface 120 is an exterior surface of the face mask object 102, or vice versa. The ultraviolet radiation system 110 may be operable to irradiate the interior surface and the exterior surface of the face mask object 102.

In the illustrated example of FIG. 1, the ultraviolet radiation system 110 is operable to irradiate the object 102 with ultraviolet radiation from a first position 122 and a second position 124 that is independent of the first position 122. In the illustrated example, a first radiation source 126 is at the first position 122 and a second radiation source 128 is at the second position 124.

In the illustrated example, the path 108 includes a conveying plane spanning a longitudinal axis 130 and a lateral axis 132. In the illustrated example, the path 108 is straight and is coincident with the longitudinal axis 130. The conveying plane has a first side 134 and second side 136 across the conveying plane from the first side 134.

In some examples, the first position 122 is adjacent the first side 134 of the conveying plane of the path 108, and the second position 124 is adjacent the second side 136 of the conveying plane of the path 108. In the illustrated example, the disinfection system 100 has a vertical axis 140, and the first radiation source 126 is above the path 108 and the second radiation source 128 is below the path 108.

The disinfection system 100 may also include a control system 142. The illustrated control system 142 may be communicatively coupled to the conveyor assembly 106 and/or to the ultraviolet radiation system 110. The control system 142 may be operable to adjust a speed at which the conveyor assembly 106 carries the object 102 along the path 108. The control system 142 may be operable to adjust a distance 144 between at least a part of the ultraviolet radiation system 110 and the object 102 as the object 102 is carried along the path 108. For example, the control system 142 may be operable to lower the first radiation source 126 and/or raise the second radiation source 128. In another example, the ultraviolet radiation system 110 may be operable to adjust the position of the conveyor assembly 106 and/or the position of one or more radiation source.

In some examples, the housing 112 is a tunnel enclosure with an open underside, and is moveable up and down relative to the base 116 received in the open underside of the housing 112. For example, the tunnel enclosure housing 112 may have an inverted u-shaped cross section. In some examples, the first radiation source 126 is mounted to the housing 112 while the second radiation source and the conveyor assembly 106 are mounted to the base 116. In some examples, the distance 144 is adjustable by moving the tunnel enclosure housing 112 up and down relative to the base 116.

The control system 142 may also be operable to adjust the output of the ultraviolet radiation system 110. For example, the control system 142 may be operable to increase or decrease the quantity of radiation emitted by an ultraviolet radiation source.

In some examples, the control system 142 is operable to receive an indication of an object type of the object 102. The control system 142 may match at least one setting to the object type, such as to adjust the ultraviolet radiation dose delivered to the object 102. For example, the control system 142 may adjust the speed of the conveyor assembly 106 to move the object 102 faster to deliver a lower dose or may adjust the speed of the conveyor assembly 106 to move the object 102 slower to deliver a higher dose. In another example, the control system 142 may decrease the distance 144 between the ultraviolet radiation system 110 and the object 102 as the object 102 is carried along the path 108 to increase the dose delivered, or may increase the distance 144 to decrease the dose.

In some examples, the control system 142 is operable to apply a plurality of settings, such as increasing the distance 144 to accommodate a larger object 102 and decreasing the speed and/or increasing the output of a radiation source to account for the increased distance 144.

In some examples, the distance 144 is at least 2 cm. In some examples, the distance 144 is at least 5 cm. In some examples, the distance 144 is at least 5 cm to provide enough space for, e.g., a face mask object 102. In some examples, the distance 144 is set to between 5 cm and 20 cm to, e.g., provide enough space for a face mask object 102.

In some examples, the control system 142 includes an imaging system 146. The imaging system 146 may be operable to generate an image of the object 102. The control system 142 may be operable to process the image to produce the indication of the object type. For example, the imaging system 146 may be positioned at a first end 148 of the tunnel of the housing 112 to generate the image when the object 102 is placed on the conveyor assembly 106 to be conveyed along the path 108. The control system 142 may receive the image, determine the object type, and adjust one or more setting to match.

In some examples, the disinfection system 100 is operable to deliver between 25 millijoules of ultraviolet C radiation per square centimeter and 1000 millijoules of ultraviolet C radiation per square centimeter to each of the at least two opposite surfaces of the object 102. In some examples, the disinfection system 100 is operable to deliver between 100 millijoules of ultraviolet C radiation per square centimeter and 1000 millijoules of ultraviolet C radiation per square centimeter to each of the at least two opposite surfaces of the object 102. In some examples, the disinfection system 100 is operable to deliver at least 100 millijoules of ultraviolet C radiation per square centimeter to each of the at least two opposite surfaces of the object 102. In some examples, the disinfection system 100 is operable to deliver at least 250 millijoules of ultraviolet C radiation per square centimeter to each of the at least two opposite surfaces of the object 102.

In some examples, only a few millijoules per square cm is needed to kill pathogens. However, in some examples, more than a few millijoules per square cm is needed to kill pathogens. In some examples, a greater dose allows for more penetration of the radiation into the object 102 and/or accounts for user error or obstructions of the radiation. In some examples, a dose above 1000 millijoules per square cm may damage some materials and/or be a wastefully high dose.

In some examples, the conveyor assembly 106 is operable to reposition the object 102 at least once as the object 102 travels along the path 108. In some examples, the conveyor assembly 106 includes a repositioning system 150 operable to reposition the object 102 at least once as the object 102 travels along the path 108. A repositioning system 150 may shift the position of the object 102 relative to the conveyor assembly 106. For example, a conveyor assembly 106 that includes a wire conveyor may block radiation from reaching portions of the object 102 that are resting on or behind wires of the wire conveyor. The repositioning system 150 may move the object 102 so that a portion of a surface of the object 102 that is positioned behind a body member of the conveyor assembly 106 relative to the ultraviolet radiation system 110 may be moved out from behind the body member.

The repositioning system 150 may be, for example, a jostling system operable to jostle the object as the object travels along the path. In the illustrated example, the repositioning system 150 is a jostling system including a compressed air nozzle 152 to direct compressed air at the object 102 as the object 102 travels along the path 108 to shift the position of the object 102. In some examples, a fan may be used to jostle the object 102. In some examples, the disinfection system 100 may include a fan or other air mover to cool the object 102.

A blocking system is another example of the repositioning system 150. The blocking system may be operable to block movement of the object 102 along the path 108 for a predetermined blocking time to reposition the object 102 relative to the conveyor assembly 106. For example, if the object 102 is held in position while an endless loop conveyor of the conveyor assembly continues moving, the object 102 will be shifted relative to the surface of the endless loop conveyor. A blocking system may include, for example, an arm or paddle removably positionable across the path 108 to obstruct movement of the object 102.

In some examples, the conveyor assembly itself is operable to reposition the object 102 at least once as the object 102 travels along the path 108. For example, the conveyor assembly 106 may include at least two staged conveyors, such as including a first endless loop conveyor operable to convey the object 102 a first way along the path 108 and then drop the object 102 onto a second endless loop conveyor to carry the object 102 the rest of the way along the path 108.

The disinfection system 100 may be powered by one or more of an external power supply and an onboard power supply. For example, the disinfection system 100 may draw power from an electrical wall outlet. In some examples, the conveyor assembly 106, the ultraviolet radiation system 110, and/or the control system 142 may be powered.

Referring now to FIG. 2, the ultraviolet radiation system may include at least one ultraviolet lamp 156. The first radiation source 126 and the second radiation source 128 may each be an ultraviolet lamp 156. In some examples, the ultraviolet lamp 156 is an ultraviolet C lamp operable to generate ultraviolet C radiation having a wavelength between 100 nanometers and 280 nanometers.

In some examples, the ultraviolet lamp 156 has an adjustable output. For example, the power or wavelength of the ultraviolet lamp 156 may be adjustable. In some examples, the control system 142 is operable to adjust the output of the ultraviolet lamp 156.

In some examples, the disinfection system 100 includes a thermal management system 161 (e.g., to control the temperature of the lamps 156). The thermal management system may include one or more heat sinks (e.g., a heat sink including a thick aluminum sheet) and/or one or more fans 163. For example, the thermal management system 161 may include at least one fan 163 for each lamp 156. In some examples, the at least one fan 163 may be used to cool the lamp(s) and/or reposition the object on the conveyor (e.g., the fan(s) 163 may be part of the repositioning system 150, such as instead of or in addition to the compressed air system and nozzle 152). For example, the at least one fan 163 may operate in at least two modes; a first mode when airflow is directed to the lamp(s) to cool the lamp and a second mode when airflow is directed to the object(s) on the conveyor to shift the object(s). The modes may differ in direction of air and/or air speed and/or air volume.

Referring now to FIG. 3, the conveyor assembly 106 may include, for example, an endless loop conveyor 158. The endless loop conveyor 158 in the illustrated example is a loop formed of open links to support the object 102 while allowing ultraviolet radiation to pass through. In some examples the endless loop conveyor 158 forms a receiving surface 160 to carry the object 102 along the path 108.

A drive motor 162 may drive the endless loop conveyor 158 at a predetermined speed. The speed may be, for example, set by the control system 142. The drive motor 162 may be powered by an external power supply or an onboard power supply.

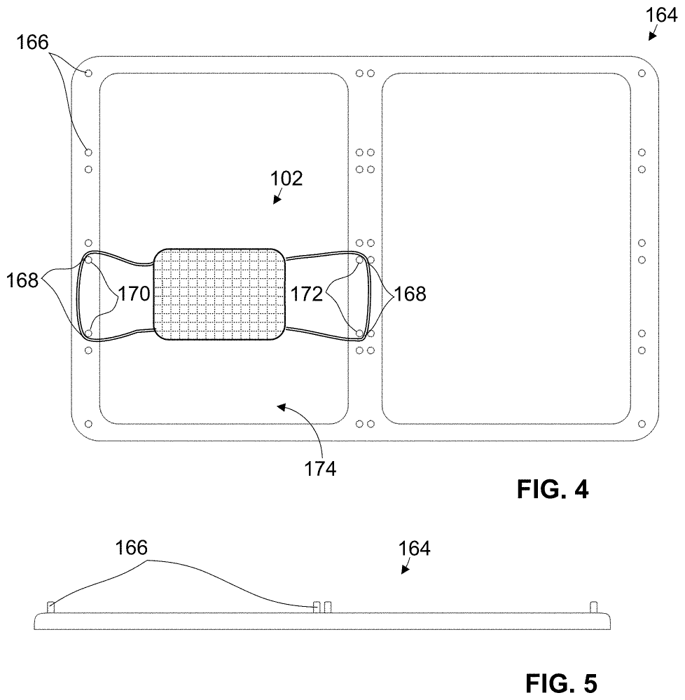

Referring now to FIGS. 4 and 5, in some examples, the disinfection system 100 includes a tray 164 to hold the object 102. In some examples, the tray 164 is shaped to hold the object 102 in an opened configuration to more easily enable the ultraviolet radiation to be delivered to the object.

In the illustrated example, the tray 164 is a removable tray. The illustrated example removable tray 164 of FIGS. 4 to 6 is shaped to hold a face mask object 102 in a fixed and opened configuration (e.g., the face mask 102 is mounted to the tray 164, and the tray 164 is shaped to hold the object 102 in a fixed position). In some examples, the face mask object 102 is an N95 mask or other shaped and/or stiff face mask. A shaped and/or stiff face mask may need to be held open by drawing two or more points (or 3 or 4 or more points) on the mask apart. The illustrated example tray 164 includes a plurality of fasteners 166 to hold the face mask object 102 at a plurality of separate points 168 (e.g., 2, 3, 4, or more points 168) on the face mask object 102. The plurality of fasteners 166 are spaced from one another so the tray 164 may hold the face mask object 102 in the opened configuration.

For example, a face mask object 102 may have a pair of elastic fastening loops 169, and one loop of the pair may be retained by a first pair of fasteners 170 while the other loop of the pair is retained by a second pair of fasteners 172 across from the first pair of fasteners 170. Where the pair of fastening loops are side loops, the face mask object 102 with the first pair of fasteners 170 and the second pair of fasteners 172 may hold the face mask object 102 in an opened configuration above an aperture 174 of tray 164.

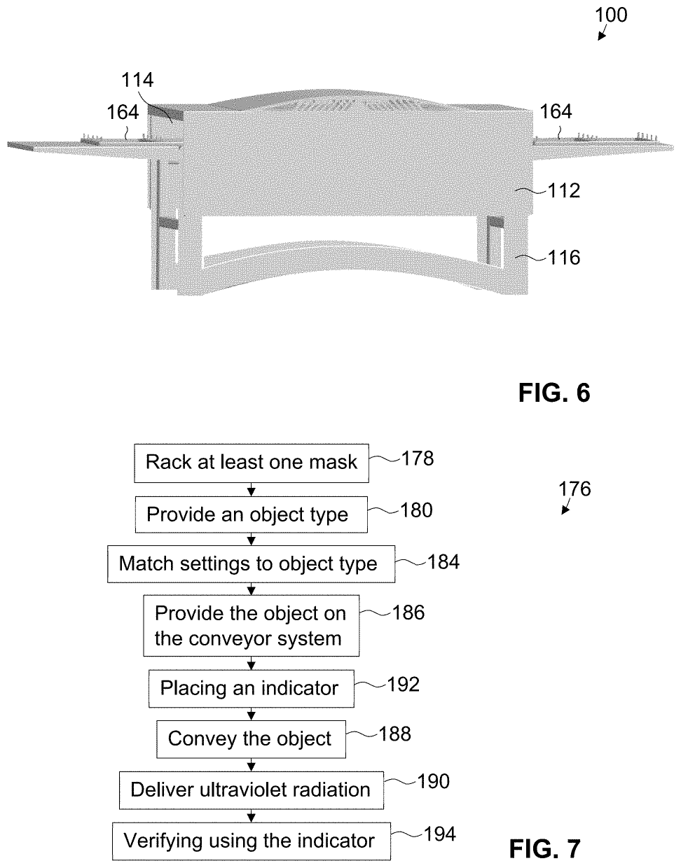

Referring now to FIGS. 6 and 7, in some examples the disinfection system 100 is used to disinfect an object 102. A method 176 of disinfecting an object 102 may include, at step 178, positioning the object 102 on a rack or tray, such as the tray 164. For example, a face mask object 102 may be stretched out and retained in position by the plurality of fasteners 166 of the tray 164.

The method 176 may then include, at step 180, providing an object type indication to the control system 142. The object type may be input by a user, such as through a touchscreen 182 (FIG. 1), or determined automatically, such as from an image captured by the imaging system 146. The control system 142 may then use the indication of object type and may, at step 184, match one or more settings of the disinfecting system 100 to the object type.

The method 176 includes, at step 186, providing the object 102 on a conveyor assembly. For example, a user may place the object 102 on a conveyer belt of the conveyor assembly 106, or the disinfection system 100 may automatically move the object 102 onto a conveyor belt of the conveyor assembly, such as from a corral or platform or other waiting area.

The method 176 includes, at step 188, conveying the object 102 along the path 108 using the conveyor assembly 106, and, at step 190, delivering ultraviolet radiation to each of two opposite surfaces of the object 102 as the object 102 is conveyed along the path 108. The ultraviolet radiation may be ultraviolet C radiation. In some examples, the method 176 includes delivering at least 250 millijoules of ultraviolet C radiation per square centimeter to each of the at least two opposite surfaces of the object 102.

In some examples, the method 176 includes delivering ultraviolet C radiation to the object 102 from at least two independent positions as the object 102 is conveyed along the path 108, the at least two independent positions including a first position 122 adjacent a first side 134 of the conveying plane and a second position 124 adjacent a second side 136 of the conveying plane across the conveying plane from the first side 122. In some examples, at least a portion of the path 108 is covered by a housing 112, and the ultraviolet C radiation is delivered to the object 102 from the at least two independent positions as the object 102 is conveyed along the path 108 within the housing 112.

In some examples, the method 176 also includes, at step 192, placing an indicator material adjacent the object 102 prior to conveying the object 102 along the path 108 using the conveyor assembly 106. The method 176 may also include, at step 194, verifying, following delivering ultraviolet radiation to each of two opposite surfaces of the object 102 as the object 102 is conveyed along the path 108, that the indicator material shows that a minimum quantity of ultraviolet radiation was delivered. In some examples, the minimum quantity of ultraviolet C radiation is 250 millijoules of ultraviolet C radiation per square centimeter.

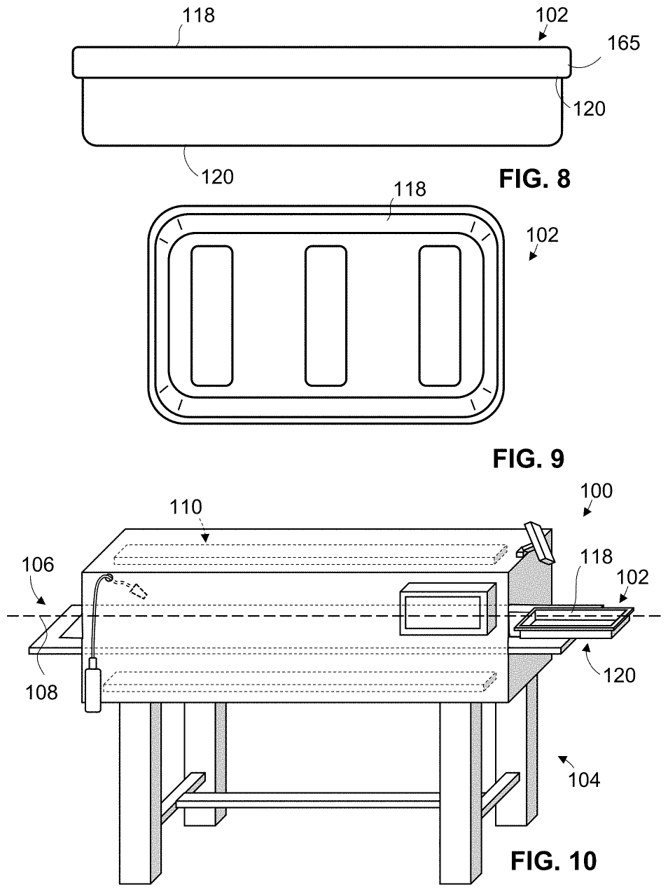

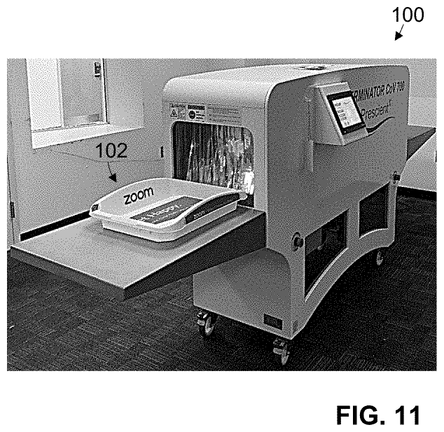

Referring now to FIGS. 8 to 11, in some examples the object 102 may be and/or include the tray 164 (e.g., a compartment tray to hold at least one item in a compartment of the tray). For example, the object may be a low-profile and open-topped compartment tray such as is used with airport security systems to hold items (e.g., luggage, keys, shoes, etc.) for inspection.

In some examples, the disinfection system 100 may be used with and/or included in an airport security system (e.g., the system 100 may include an x-ray scanner or other sensor(s) mounted to frame 104).

In some examples, the first surface 118 is an interior surface of the tray 164 and the second surface 120 is an exterior surface of the tray 164 (e.g., including an underside of a peripheral rim 165), or vice versa. The ultraviolet radiation system 110 may be operable to irradiate the interior surface and the exterior surface of the tray 164.

The present invention has been described here by way of example only. Various modification and variations may be made to these exemplary embodiments without departing from the scope of the invention, which is limited only by the appended claims.

1. A disinfection system for disinfecting an object using ultraviolet radiation, comprising:
a conveyor assembly operable to carry the object along a path, the path defining a conveying plane;
a support frame supporting the conveyor assembly;
an ultraviolet radiation system mounted to the support frame and operable to irradiate the object with ultraviolet radiation from at least two independent positions as the object is carried along the path, the at least two independent positions including a first position adjacent a first side of the conveying plane and a second position adjacent a second side of the conveying plane across the conveying plane from the first side.

2. The disinfection system of claim 1, wherein the support frame includes a housing covering at least a portion of the path, and the ultraviolet radiation system is operable to irradiate the object with ultraviolet radiation from the at least two independent positions as the object is carried along the path within the housing.

3. The disinfection system of claim 2, wherein the housing includes a tunnel enclosure through which the path extends.

4. The disinfection system of claim 3, wherein at least one inner surface of the housing is a reflective surface.

5. The disinfection system of claim 1, wherein the ultraviolet radiation system includes at least two independent radiation sources.

6. The disinfection system of claim 5, wherein each of the at least two independent radiation sources is an ultraviolet C lamp operable to generate ultraviolet C radiation.

7. The disinfection system of claim 1, wherein the conveyor assembly includes an endless loop conveyor forming a receiving surface to carry the object along the path.

8. The disinfection system of claim 1, further comprising a control system operable to adjust at least one of a speed at which the conveyor assembly carries the object along the path and a distance between the ultraviolet radiation system and the object as the object is carried along the path,

9. The disinfection system of claim 8, wherein the control system is operable to receive an indication of an object type of the object and match at least one of the speed and the distance to the object type.

10. The disinfection system of claim 9, wherein the control system includes an imaging system operable to generate an image of the object, and the control system is operable to process the image to produce the indication of the object type.

11. The disinfection system of claim 1, wherein the object includes a removable compartment tray that may be removably positioned on the conveyor assembly to be carried along the path while holding at least one item in a compartment of the removeable compartment tray.

12. The disinfection system of claim 1, wherein the object includes a face mask, and the face mask is mounted to a removeable fixation tray, and the removable fixation tray is operable to carry the face mask in a fixed and opened configuration.

13. The disinfection system of claim 12, wherein the removable tray includes a plurality of fasteners to hold the face mask at a plurality of separate points on the face mask, the plurality of fasteners spaced from one another to hold the face mask in the opened configuration.

14. The disinfection system of claim 1, wherein the ultraviolet radiation system is operable to deliver at least 250 millijoules of ultraviolet C radiation per square centimeter to each of at least two opposite surfaces of the object.

15. The disinfection system of claim 1, wherein the conveyor assembly is operable to reposition the object at least once as the object travels along the path.

16. The disinfection system of claim 15, wherein the conveyor assembly includes at least one of a jostling system operable to jostle the object as the object travels along the path and a blocking system operable to block movement of the object along the path for a predetermined blocking time to reposition the object relative to the conveyor assembly.

17. The disinfection system of claim 1, wherein the object includes a face mask and the ultraviolet radiation system is operable to deliver ultraviolet radiation to at least two opposite surfaces of the face mask, the at least two opposite surfaces of the face mask including an exterior surface of the face mask and an interior surface of the face mask.

18. A disinfection method for disinfecting an object using ultraviolet radiation, comprising:
placing the object on a conveyor assembly;
conveying the object along a path using the conveyor assembly, the path defining a conveying plane; and
delivering ultraviolet C radiation to the object from at least two independent positions as the object is conveyed along the path, the at least two independent positions including a first position adjacent a first side of the conveying plane and a second position adjacent a second side of the conveying plane across the conveying plane from the first side.

19. The disinfection method of claim 18, further comprising:
placing an indicator material adjacent the object prior to conveying the object along the path using the conveyor assembly;
conveying the indicator material with the object along the path; and
verifying, following delivering ultraviolet C radiation to the object, that the indicator material shows that a minimum quantity of ultraviolet C radiation was delivered.

20. The disinfection method of claim 18, wherein at least a portion of the path is covered by a housing, and the ultraviolet C radiation is delivered to the object from the at least two independent positions as the object is conveyed along the path within the housing.
